(19) United States
(12) Patent Application Publication
(10) Pub. No.: US 2021/0056496 A1
(43) Pub. Date: Feb. 25, 2021

(54) SYSTEM FOR FACILITATING PURCHASE OF PRESCRIPTION DRUGS

(71) Applicants: David Edward Gajeski, Katy, TX (US); James Charles Adcox, Magnolia, TX (US)

(72) Inventors: David Edward Gajeski, Katy, TX (US); James Charles Adcox, Magnolia, TX (US)

(21) Appl. No.: 16/546,866

(22) Filed: Aug. 21, 2019

(51) Int. Cl. G06Q 10/08; G16H 20/10; G16H 80/00

(52) U.S. Cl. CPC G06Q 10/087; G06Q 10/0832; G16H 80/00; G16H 20/10; G06Q 10/0833

(57) ABSTRACT

A computer system to assist in distribution of prescription drugs is provided. Prescription drug distribution is a highly regulated and controlled supply chain. Consumers often visit a Pharmacy to fill doctor prescriptions. Payment amounts for prescriptions vary based on health plan coverage negotiated prices. Drugs are made by a Manufacturer but may transition through a Distributor or wholesaler on their way to Pharmacies (where consumers may ultimately make a purchase for use). Pharmacies, Distributors, and Manufacturers keep an inventory of drugs to meet supply demands. Regulations and contracts may prevent transition between stages of a distribution chain. This prevention may be artificial in that the drugs, if allowed to proceed, would still be valid and usable by a consumer. When transition is prevented drugs may be placed in a Drug Morgue and then sold from the Drug Morgue to prevent destruction or other form of handling resulting in wasted inventory.

### BACKGROUND

Consumers often visit a Pharmacy to fill prescriptions issued by their doctor. The amount a Consumer pays for a prescription may vary based on prices previously negotiated by health plan coverage. The drugs used to fill prescriptions are made by a Manufacturer that may use a Distributor or Wholesaler as the method to deliver the drugs to multiple Pharmacies. Pharmacies, Distributors, and Manufacturers each maintain an inventory of drugs available to meet the purchasing demands of the Consumers that need to fulfill prescriptions.

Drugs, like food, have an expiration date after which they should be discarded. The length of time before a drug expires may depend on the type of drug and storage environmental conditions. Manufacturers and Distributors may proactively send quantities of drugs to Pharmacies based on standing contract quantities per time period but typically Pharmacies order drugs as needed. Several factors may dictate a minimum timespan (e.g., “usability period”) between the current date and the expiration date of a drug before a drug, though it may still be consumable, may not be sent to a Pharmacy as part of a shipment of drugs.

For example, regulations or contract stipulations may dictate that a drug with a 2-year shelf-life may be sent to a Pharmacy as part of a shipment up until 1 year before the drug expires. These time periods may vary for different drugs and this 2-year/1-year time duration is purely illustrative. Drugs that may not be acceptable (e.g., based on purchase contract terms) to be sent to a Pharmacy (but may still be good for consumption) are typically placed in a “Drug Morgue” (“DM”). A DM may be maintained by any party within the distribution chain including the Manufacturer or Distributor. Thus, useable drugs may be placed in a DM even though they may still have a viable use. This disclosure provides solutions to these and other problems, in part, by enhancing existing distribution channels for prescription drugs and introducing new controlled distribution possibilities.

### BRIEF DESCRIPTION OF THE DRAWINGS

The present disclosure may be better understood from the following detailed description when read with the accompanying Figures. It is emphasized that, in accordance with standard practice in the industry, various features are not drawn to scale. In fact, the dimensions or locations of functional attributes may be relocated or combined based on design, security, performance, or other factors known in the art of computer systems. Further, order of processing may be altered for some functions, both internally and with respect to each other. That is, some functions may not require serial processing and therefore may be performed in an order different than shown or possibly in parallel with each other. For a detailed description of various examples, reference be made below to the accompanying drawings, in which:

FIG. 1 illustrates an example drug product distribution flow showing product, payment, and rebate flow that may be improved using an inventory control system that interacts with different actors across a prescription drug product distribution flow, according to one or more disclosed implementations;

FIG. 2 illustrates a diagram of an example system that may be used to facilitate the product, payment, distribution, and rebate flow of FIG. 1, according to one or more disclosed implementations;

FIG. 3 illustrates a diagram showing entity interaction with possible implementations of the system of FIG. 2, according to one or more disclosed implementations;

FIG. 4 illustrates an example system component diagram showing possible functional components of an improved inventory control system for prescription drug distribution flow, according to one or more disclosed implementations;

FIG. 5 illustrates a process flow for a system (e.g., an enhanced inventory control system) facilitating the product, payment, distribution, and rebate flow, according to one or more disclosed implementations;

FIG. 6 illustrates an example computing device, with a hardware processor, and accessible machine-readable instructions stored on a machine-readable medium that may be used to implement a system for facilitating the purchase and distribution of prescription drugs, according to one or more disclosed example implementations;

FIG. 7 presents a computer network infrastructure that may be used to implement all or part of the disclosed techniques for a central system receiving and recording updates to inventory or purchase requests to facilitate prescription drug purchases and inventory control, according to one or more disclosed embodiments;

FIG. 8 illustrates a computing device that may be used to implement the functions, modules, processing platforms, execution platforms, communication devices, and other methods and processes of this disclosure.

FIG. 9A illustrates a drug lifecycle with respect to a Manufacturer, according to one or more disclosed embodiments; and

FIG. 9B illustrates a drug lifecycle with respect to a wholesaler, according to one or more disclosed embodiments.

### DETAILED DESCRIPTION

Illustrative examples of the subject matter claimed below will now be disclosed. In the interest of clarity, not all features of an actual implementation are described for every example implementation in this specification. It will be appreciated that in the development of any such actual example, numerous implementation-specific decisions may be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

As briefly explained above, in a distribution flow for prescription drugs a holding area referred to as a “Drug Morgue” (“DM”) may exist at different points in the distribution chain. Drugs that are placed in a DM may still be safely consumed by Consumers filling prescriptions (i.e., the drugs have not yet expired but may have a shortened length of use till expiration). In some cases, drugs in a DM may not be sent proactively to a Pharmacy but may be directly requested by a Pharmacy in cases where the Pharmacy may expect the drugs to be dispensed and consumed before the expiration date. Manufacturers and Distributors may have an incentive to lower the price of drugs in the DM (e.g., because of their limited use period). A Pharmacy may know the demand for drugs and can assess if prescriptions may be filled by drugs in a DM based on consumer demand. A Pharmacy may, for example, request drugs from the Manufacturer's or Distributor's DM to increase their profit margin on dispensed prescriptions. A system for cataloging drugs in a DM and allowing purchase of these drugs is disclosed.

The system may allow one or more persons representing a Manufacturer, a Distributor, a Pharmacy, a Prescription Benefits Manager, or any other role in the prescription drug supply chain create a user account with associated contact information. Additional information to properly and legally dispense a controlled substance may also be provided. Different roles may have different forms of associated information with respect to each role.

For example, a Pharmacy may supply a DEA number, a Pharmacy number, or other information that may be used to verify the entity signing up for the role in the prescription drug supply chain may legitimately function in that role. In general, this may be considered a purchasing entity registering with the system so that they may obtain access to and use of features of the disclosed prescription drug purchase system. Some examples of other types of information that may be maintained within the disclosed system include a Pharmacy type, an annual spend amount, and contact information. A Pharmacy type may include retail, such as a neighborhood Pharmacy, or an acute Pharmacy, such as within a hospital or associated with an urgent care clinic. An acute Pharmacy typically has different stocking requirements than a retail Pharmacy due to the nature of client they support. For example, at a retail Pharmacy it may not be an issue to have a customer wait a day or two (or even longer) to have a prescription filled. In contrast, an acute Pharmacy may not allow for delay in filling prescription requests. As a result, an acute Pharmacy may have more “aggressive” stocking requirements for certain drugs and be subject to potential additional inefficiencies that make their DM more active.

In some instances, a user account may be placed in a temporary hold state where the user(s) of the account have no access to participate in activities provided by the system until the account is verified. The verification may occur through automated data exchange with external systems such as, for example, a database (e.g., a database of the Drug Enforcement Agency (“DEA”) or other authorization entity). In any case, the database may include information about entities that are authorized to receive and dispense drugs classified as controlled substances. Different user accounts may have different classifications as to the amount, type, or time period for which they are allowed to distribute (e.g., sell) different classifications of drugs (e.g., based on a drug classification schedule). The verification may also include a manual verification through interaction between the entity creating the account and any other external entities that may assist in account validation.

Users that maintain accounts in the role of Manufacturer or Distributor may create, update, or delete inventory records of drugs or other items that may be offered for sale. This information may include, for example, the name of the item, pictures of the item, description of the item, price of the item, useable term for the item, minimum purchase quantities, disclosures of side-effects or dangerous interactions with other drugs, payment terms, shipping cost, or any other information used to facilitate the sale of the item. Multiple prices may be included that, for example, determine a per-unit price based on an order quantity, a per-unit price for drugs that are in the DM, or any other logic that facilitates the purchase of an item. The item information may also be updated to have dynamic pricing and payment terms where in some examples a buyer is given contact information for a representative of a Manufacturer or Distributor to negotiate a per-unit price. In some cases, dynamic pricing may be implemented to automatically lower a price of a drug with respect to a decrease in the remaining viable time period of the drug prior to expiration of that drug.

The Manufacturer or Distributor may also utilize data entered for items for sale to support an auction type of sale as an example. Any type of sale may be supported and utilize different credit purchase types or payment methods including cryptocurrency. The Manufacturer or Distributor may set criteria for the auction such as starting price, minimum sale price, the time length in which bids are accepted, or any other method that may be used to ensure the sale terms of the item are suitable to the Manufacturer or Distributor at the end of the auction. System users in roles (e.g. the Pharmacy role) may offer a bid in competition with other system users that conforms to the auction rules configured by the Manufacturer or Distributor. At the end of the auction, the system user with the highest bid price is expected to present payment (how the system facilitates multiple payment methods is described later) to obtain the items purchased via auction.

The system may also provide the user a method to update available inventory quantities either through a manual input (e.g. the user navigates to each item individually and updates the quantity on hand). The system may also provide the capability to update available inventory through an automated data exchange such as uploading a Comma Separated Value (“CSV”) file, using an Application Programming Interface (“API”), or using any other method of data exchange to facilitate the update of inventory quantity. Interfaces to point of sale (“POS”) systems may be included to maintain inventory information as of each consumer sale.

Users representing a Manufacturer may have the previously described capabilities and may have additional or different capabilities. Specifically, an entity participating in the role of Manufacturer may choose to sell drugs directly to other entities in the system or it may choose to create item records that direct entities attempting to purchase items to a Distributor for one or more items. The Manufacturer may configure the item record to have contact information (e.g. phone number, email, fax, etc.) for one or more Distributors. Using this type of information, when a purchaser selects an item for purchase, the purchaser may be presented with associated contact information to complete the purchase.

Traditional purchase practices may include a user participating in the role of Benefits Manager. However, different implementations of disclosed systems may attempt to eliminate or reduce the role of a Benefits Manager by streamlining the prescription drug procurement process according to disclosed embodiments. If implemented, the Benefits Manager may also create an account as described previously, may create, update, or delete data that indicates contractual pricing for items offered for purchase by entities in the Manufacturer or Distributor role. The information maintained by the Benefits Manager may include a name of a benefits plan, description of the prescription drug consumer eligibility, and any other information that may be used to give a consumer of prescription drugs. In addition, a discounted price may be determined that is based on a negotiated purchase price between the Benefits Manager and the Manufacturer or Distributor. The information provided by the Benefits Manager may be used by other users of the system that are in other roles to identify consumers that are valid members of one or more benefits plans configured by different Benefits Managers.

A user in the role of Pharmacy may utilize a search capability to view items that are offered for sale by users or entities associated with the roles of Manufacturer or Distributor. Any data entered into the system (as described previously) may be displayed as part of the search results. The user in the role of Pharmacy may filter the results by any criteria related to the data displayed for each item for sale. The user in the role of Pharmacy may, for example, search for a drug by name with a unit price within a user-defined range while also indicating they would accept drugs from the Manufacturer or Distributor's DM that have a specific duration of useable term (e.g., time till expiration).

The search results may display results that are color coded to indicate a variable availability based on the inventory information provided by a Manufacturer or Distributor. A search result for a drug may be colored green, for example, if the expiration date is far in the future or the quantity available is very large. A yellow search result may indicate that the drug's date in which it must be placed in the Manufacturer's or Distributor's morgue is approaching. A red search result may indicate that the drug is in the DM or the available quantity is low. These examples of color coding are not intended to limit the methods of determining colors or the meaning of the colors. The number of colors used and how to color the search results may be implemented with any logic, number of colors, or intended meaning for a particular color.

A user in the role of Pharmacy may browse items for sale offered by Manufacturers or Distributors and select items for purchase. The items may or may not be drugs that are in a DM. Next, the user may select items based, in part, on a status tag that indicates (e.g., via visual representation) if that item is a DM item or if the drug's expiration date does not qualify it as a DM item. The system may also predict when certain lots of drugs will be classified as a DM item. Thus, an item may be a short time period away from entering a DM classification and the purchase may be delayed for that short time period (or the price negotiated) to facilitate purchase of that lot.

Based on the item's purchasing terms (e.g., as configured by the Manufacturer or Distributor), the user may select the item for immediate or future purchase. For immediate purchases, the system may facilitate payment for purchases in a variety of ways. In one example, the system may collect an electronic payment made by a credit card, debit card, bank wire transfer, or any other method of electronic payment including cryptocurrency exchange. In another example, the user may be presented an invoice electronically or by regular mail to be paid based upon payment terms dictated by the Manufacturer or Distributor. For future purchases, the purchase in one example may be a one-time purchase dated for the future in which payment is collected using the same methods as described for immediate purchases. In another example of a future purchase, the user may indicate a reoccurring order with a reoccurrence time period (e.g. the order may reoccur, for example, on the first day of every month). The payment for reoccurring orders may be collected using the same methods as described for immediate purchases. Future or seasonal purchases may be applicable to certain types of drugs. For example, each year a new version of a Flu vaccine is typically made available. For these types of drugs, a retail Pharmacy may negotiate a purchase price prior to the season (or time period) for which the drug is applicable.

A user in the role of Pharmacy may use a background search capability to be alerted when, for example, new items are added for sale by Manufacturers or Distributors, prices change on items indicated in the search criteria, or any other applicable criteria that may be used to facilitate alerting the Pharmacy of item status. Specifically, a Pharmacy user may be informed (e.g., via alert) that certain drugs have entered a DM classification (e.g., a DM state at a different point in the supply chain). Alerts may be created, updated, and deleted by the user at any time.

A user in the role of Pharmacy may create, update, or delete requests to purchase items that may be viewed by other system users. The Pharmacy role user may specify the item they wish to purchase, the quantity they wish to have fulfilled, the per-unit price they are willing to pay, and any other information used to describe the item and terms of purchase desired. Other system users may contact the user in the role of Pharmacy to offer to fulfill their request or offer an alternate higher price in which the request may be fulfilled. This on-demand purchase request capability may be used by the system users to facilitate a negotiation of a price that one user is will pay to another user to fulfill an order for the item.

A user in the role of Pharmacy may interface the system with their POS system or any other system the Pharmacy may use to sell dispense prescriptions to consumers. As part of the interfacing, benefit plan information entered by the Benefits Manager may be utilized by the POS to adjust the price of a prescription based on benefits plans in which the consumer is a participant. The POS may collect information requested from the consumer to validate that the consumer is a valid member of a benefits plan accepted by the Pharmacy.

In some implementations, a class of trade (“COT”) with respect to the Pharmacy type (e.g., acute, retail, etc.) may be used by a wholesaler or Manufacturer may be a factor utilized to determine price. In alternate implementations, the COT may simply be ignored.

The system, when facilitating purchases of items using any method described above, may have the ability to inform system users of the status of their orders. A seller may update the system when items are shipped to a buyer. Shipment updates may be delivered to the buyer via email, Short Message Service (“SMS”) text, or any other method of delivering information to the buyer. Buyers may track the status of their orders in the system by viewing the state of the order as the state data is updated by a seller. Sellers may enter tracking numbers provided by companies that may be transporting the shipment, and a buyer may be directed to an external system to view the tracking information for the shipment via the tracking number provided to the seller upon shipment of the order.

The system may provide reporting on metrics collected during the operation of the system. In one example, a report may be generated showing the display of times an item for sale is viewed by a system user with the report breaking down the counts by each Manufacturer or Distributor. In another example, a report displaying the total amount of money paid may be displayed with subtotals broken down by currency, Manufacturer, Distributor, product class, or any combination of grouping. The reporting capability may allow a user to configure how to group data by pivoting known data points by rows and columns (e.g., similar to a pivot table function of a spreadsheet).

Some reports may be limited to display data related to their own activity on the system. For example, a Manufacturer or Distributor may be able to show a report of total amounts paid to them by Pharmacies but may not be able to see the amounts the same Pharmacies paid to other Manufacturers or Distributors. A system administrator, however, may be able to see the same report showing totals of amounts paid by Pharmacies broken down by all Manufacturers or Distributors. In general, different levels of security and visibility to data of different users and user roles may be provided.

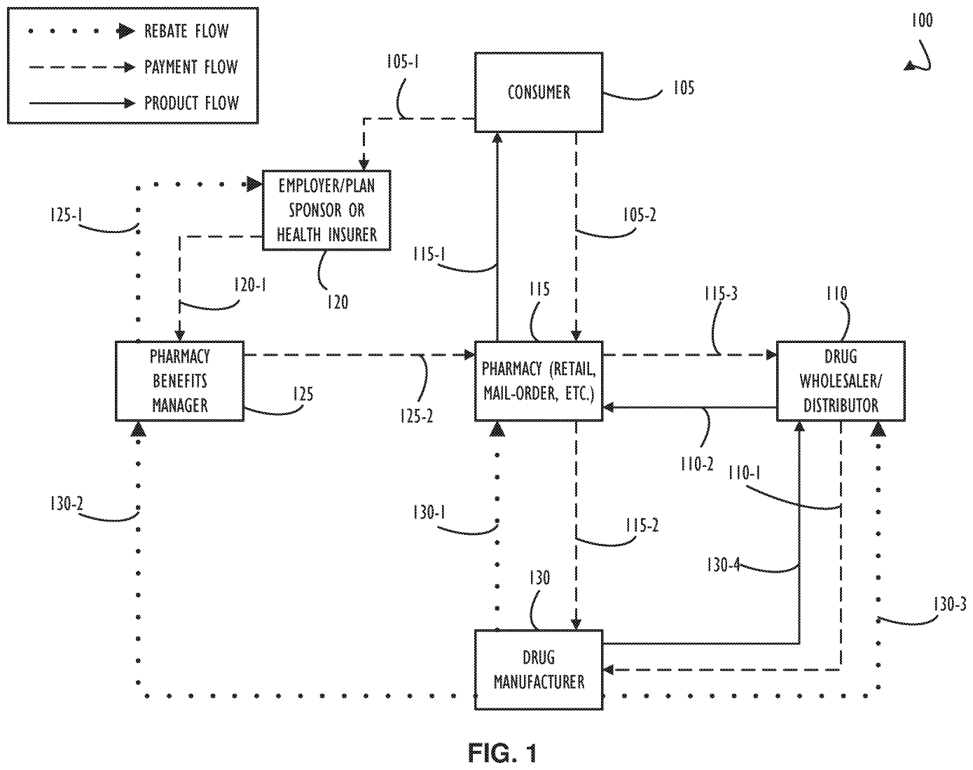

Referring now to FIG. 1, shown is an example topology map 100 showing product, payment, and rebate flow. Each of the connection points in topology map 100 may indicate a point of data exchange (e.g., network data transaction) between functional modules of the disclosed computer-based system. Further, the different functional modules may be distributed across computers physically at different locations (e.g., Pharmacy, Manufacturer, etc.) or may be provided at a central server. In one example, a cloud-based system may be implemented to support disclosed systems without having physical dedicated hardware at different locations. In any case, one of ordinary skill in the art, having the benefit of this disclosure, will recognize that a comprehensive computer system may be developed to provide the techniques of this disclosure.

In FIG. 1, consumer 105 is illustrated to participate in a prescription benefits plan offered by a health insurer 120. Consumer 105 may pay to participate in the health plan, as indicated by the connecting line 105-1 (each connecting line in FIG. 1 may represent a transaction that includes automated data exchange). When consumer 105 visits Pharmacy 115 to fill a prescription, consumer 105 may pay a discounted rate for the prescription as indicated by connecting line 105-2. Pharmacy benefits manager 125 may pay Pharmacy 115 to subsidize purchase by consumer 105, as indicated by connecting line 125-2. Consumer 105 may then receive the dispensed drugs from the Pharmacy 115 as indicated by connecting line 115-1.

The currency (form of payment) subsidizing purchase by consumer 105 that may be provided from benefits manager 125 may be allocated to benefits manager 125 as a result of health plan 120, as indicated by connecting line 120-1. Rebates from drug Manufacturer 130 may also be provided to benefits manager 125, as indicated by connecting line 130-2. A portion of the rebates received by benefits manager 125 may be passed on to health insurer 120 as indicated by connecting line 125-1.

Pharmacy 115 may also pay Manufacturer 130, for example, if they are buying directly from Manufacturer 130. Pharmacy 115 may also receive rebates from the Manufacturer 130, as indicated by connecting line 130-1. For example, rebates may be offered instead of a lower price. Pharmacy 115 may purchase drugs used for filling prescriptions from Distributor 110, where the purchase is indicated by connecting line 115-3 and receipt of the drugs is indicated by connecting line 110-2. Distributor 110 may receive the drugs from Manufacturer 130, as indicated by connecting line 130-4, after making a payment to Manufacturer 130 as indicated by connecting line 110-1. Manufacturer 130 may also pay rebates to Distributor 110, as indicated by connecting line 130-3. For example, rebates may be paid as part of price negotiation or due to volume tiers with respect to purchases in a time period. Specifically, a volume purchaser that procures more inventory may receive a discount based on a volume tier for which that purchaser qualifies.

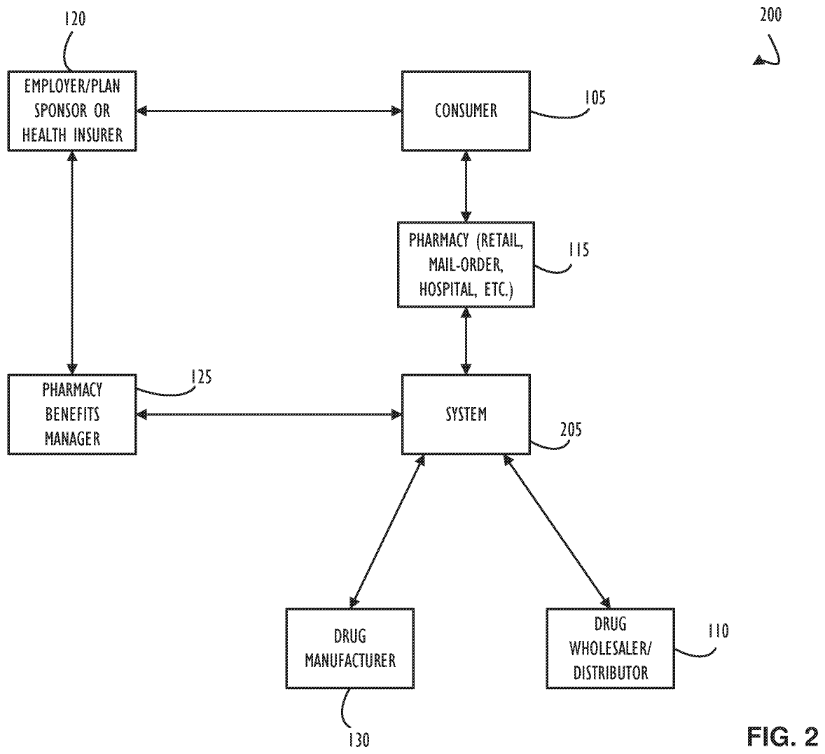

Referring now to FIG. 2, shown is an example diagram of a system 205 facilitating product, payment, and rebate flow 200. System 205 is a high-level representation that may be composed of both server and client applications that allow the storage of data collected through interactions with users and systems owned by users. For example, Distributor 110 and Manufacturer 130 are shown as being able to interact with system 205. Interactions may be to enter information about items offered for sale, or for other interactions as described above. For example, additional interactions by Distributor 110 and Manufacturer 130 may be to configure pricing information negotiated for health plans that may be associated with one or more Pharmacy benefits managers 125.

Pharmacy 115 may also interact with system 205, for example, to search for items to purchase offered by Distributor 110 and Manufacturer 130, as described above. Pharmacy 115 may also utilize health plan information entered into system 205 by Pharmacy benefits manager 125. Pharmacy 115 may also utilize system 205 when interacting with consumer 105 to set a price for which consumer 105 pays to receive a dispensed prescription. In some instances, consumer 105 may belong to a health plan 120 that subsidizes the price for different consumers 105 with respect to the dispensed prescription (e.g., via negotiated prices that may have been facilitated by benefits manager 125).

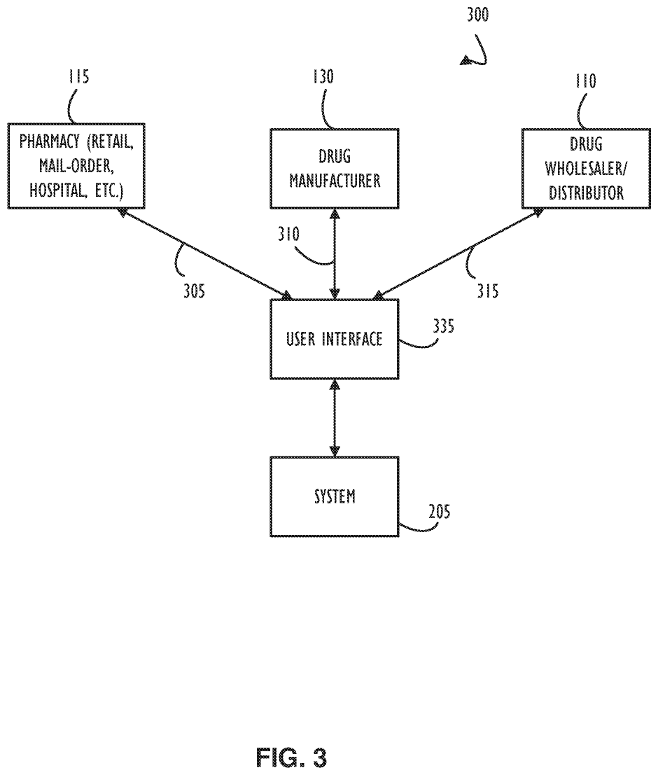

Referring now to FIG. 3, shown is a diagram showing entity interaction flows 300 with system 205. System 205 may have a variety of interfacing methods, as indicated by different ones of flows 300, including a user interface 335 that may allow entities to create, update, and delete data from the system as part of facilitating the product, payment, and rebate flow 100 as illustrated in FIG. 1.

For example, Distributor 110 may perform interaction 315 with the user interface 335 to enter information for items for sale. Manufacturer 130 may also perform interaction 310 with user interface 335 to enter items for sale, which may include additional information such as directing purchase inquiries to a Distributor 110. Pharmacy 115 may perform interaction 305 with user interface 335 to view and purchase items for sale.

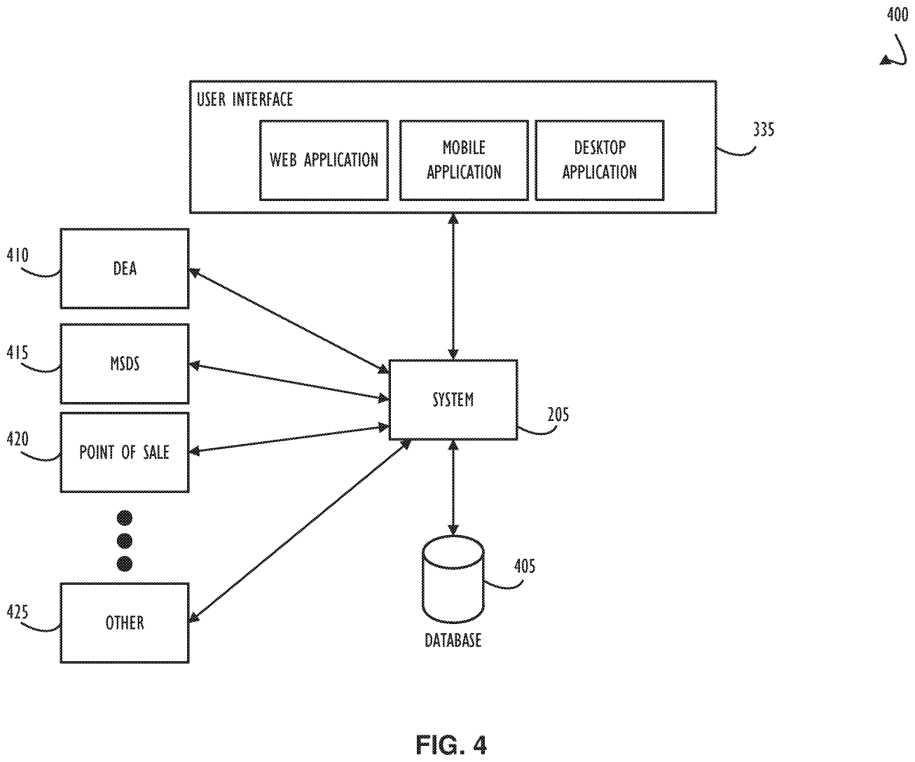

Referring now to FIG. 4, shown is an example system 400 is illustrated as a functional component block diagram. A variety of user interfaces 335 may be provided by system 205, such as a web interface, a mobile application interface, or a desktop application interface. User interface 335 may also provide a means to for entities to create, update, and delete data from system 205 as part of facilitating the product, payment, and rebate flow 100 as illustrated in FIG. 1.

System 205 may utilize a database 405 or other data store to store created data, store data updates, and remove data that may be initiated by actions of entities via any user interface 335. Database 405 may also be utilized to retrieve data displayed in user interface 335 in response to actions (such as the previously described search or reporting) taken by entities utilizing the user interface 335. Database 405 may be implemented in a variety of ways and include a central or distributed database. Database 405 may include a variety of data storage techniques and repositories including: relational databases, extensible markup language (“XML”) files, CSV files, and the like. Note that other implementations may use other types of data stores besides databases.

System 205 may perform automated interactions through APIs (not shown) to other external systems (not all shown) as part of the logic implemented by system 205. For example, a DEA Database 410 may be used to validate the ability of a Pharmacy to receive and dispense controlled substances. In another example, system 205 may utilize a Material Safety Data Sheet (“MSDS”) database 415 to allow entities utilizing user interface 335 to see MSDS specifications for drugs. System 205 may interface with a POS system 420 to assist with setting the price a consumer must pay for a dispensed prescription. An unlimited number of other external systems 425 (indicated by the ellipses) may be utilized to facilitate the product, payment, and rebate flow 100 as illustrated in FIG. 1.

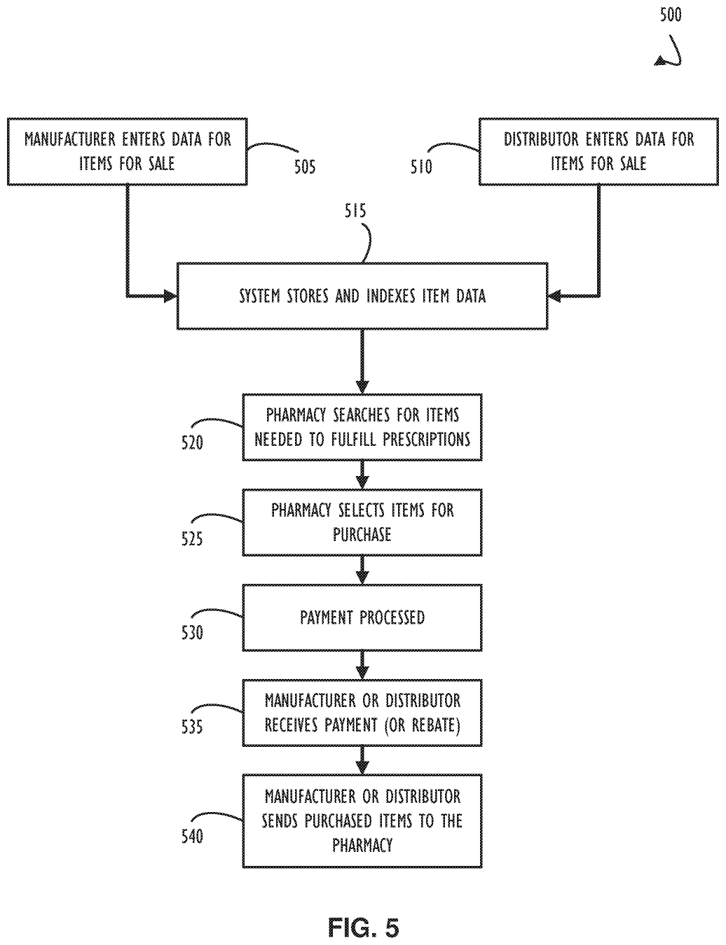

Referring now to FIG. 5, shown is a process flow for the system facilitating the product, payment, and rebate flow as an example method 500. Method 500 begins at block 505 or 510, where a Manufacturer (e.g., Manufacturer 130) may enter data into the system (indicated in block 505) or a Distributor (e.g., Distributor 110) may enter data into the system (indicated in block 510). The entered data may, for example, be data about items being offered for sale. Flow continues to block 515 where the system may record and index the data entered.

Flow continues to block 520 where a Pharmacy (e.g., Pharmacy 115) may search the entered data for items to purchase. Flow continues to block 525 where the Pharmacy selects items to purchase, using methods such as those described above. Flow continues to block 530 where a payment is processed for the items selected for purchase. Purchase methods may include direct payment between the entity making the purchase and the entity making the sale. The system may also facilitate a payment between the entity making the purchase and the entity making the sale. Continuing to block 535, the Manufacturer or Distributor receives the payment made by the Pharmacy. Flow then continues to block 540 where the Manufacturer or Distributor, having received a payment facilitated by the system, sends the purchased items to the Pharmacy.

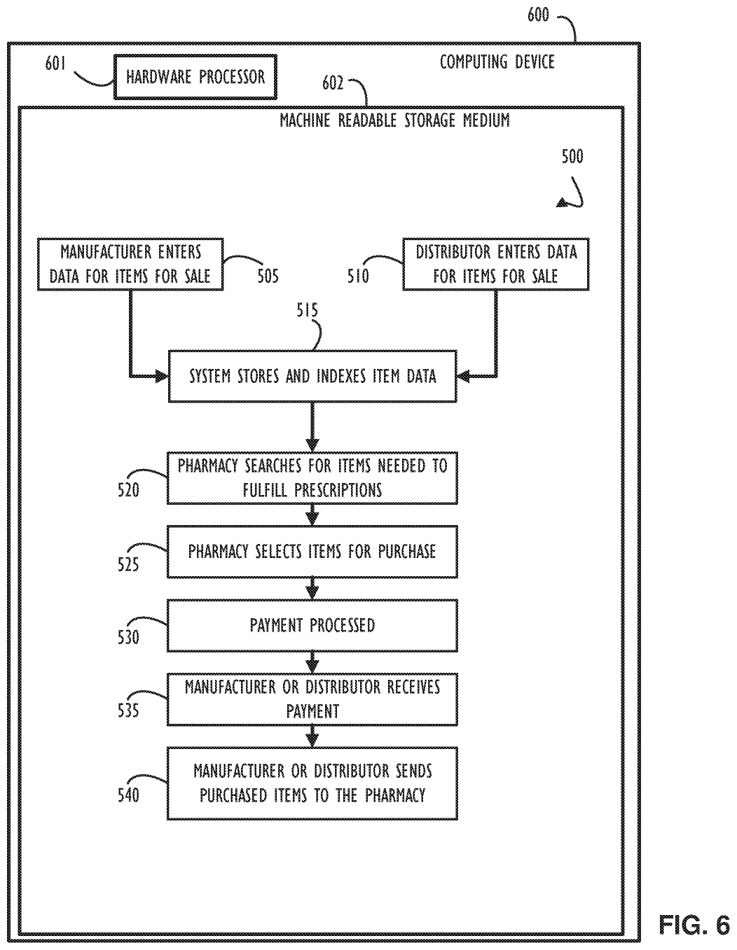

Referring to FIG. 6, shown is an example computing device 600, with a hardware processor 601, and accessible machine-readable instructions stored on a machine-readable medium 602 that may be used to implement the system for facilitating the purchase and distribution of prescription drugs, according to one or more disclosed example implementations. FIG. 6 illustrates computing device 600 configured to perform the flow of method 500 as an example. However, computing device 600 may also be configured to perform the flow of other methods, techniques, functions, or processes described in this disclosure. In this example of FIG. 6, machine-readable storage medium 602 includes instructions to cause hardware processor 601 to perform blocks 505-540 discussed above with reference to FIG. 5.

A machine-readable storage medium, such as 602 of FIG. 6, may include both volatile and nonvolatile, removable and non-removable media, and may be any electronic, magnetic, optical, or other physical storage device that contains or stores executable instructions, data structures, program module, or other data accessible to a processor, for example firmware, erasable programmable read-only memory (“EPROM”), random access memory (“RAM”), non-volatile random access memory (“NVRAM”), optical disk, solid state drive (“SSD”), flash memory chips, and the like. The machine-readable storage medium may be a non-transitory storage medium, where the term “non-transitory” does not encompass transitory propagating signals.

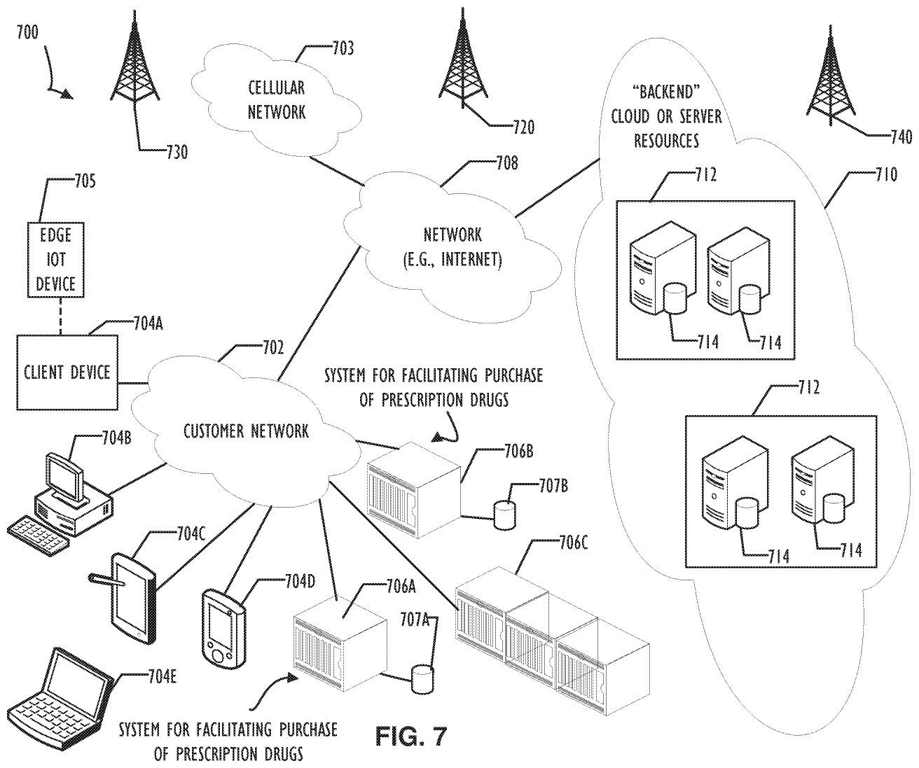

FIG. 7 represents a computer network infrastructure 700 that may be used to implement all or part of the disclosed the system for facilitating the purchase and distribution of prescription drugs, according to one or more disclosed implementations. Network infrastructure 700 includes a set of networks where implementations of the present disclosure may operate in one or more of the different networks. Network infrastructure 700 comprises a customer network 702, network 708, cellular network 703, and a cloud service provider network 710. In one implementation, the customer network 702 may be a local private network, such as local area network (“LAN”) that includes a variety of network devices that include, but are not limited to switches, servers, and routers.

Each of these networks may contain wired or wireless programmable devices and operate using any number of network protocols (e.g., TCP/IP) and connection technologies (e.g., WiFi® networks, or Bluetooth®. In another implementation, customer network 702 represents an enterprise network that could include or be communicatively coupled to one or more local area networks (“LANs”), virtual networks, data centers and/or other remote networks (e.g., 708, 710). In the context of the present disclosure, customer network 702 may include one or more high-availability switches or network devices using methods and techniques such as those described above (e.g., system for facilitating purchase and distribution of prescription drugs as shown for compute-resource 706A and compute-resource 706B).

As shown in FIG. 7, customer network 702 may be connected to one or more client devices 704A-E and allow the client devices 704A-E to communicate with each other and/or with cloud service provider network 710, via network 708 (e.g., Internet). Client devices 704A-E may be computing systems such as desktop computer 704B, tablet computer 704C, mobile phone 704D, laptop computer (shown as wireless) 704E, and/or other types of computing systems generically shown as client device 704A.

Network infrastructure 700 may also include other types of devices generally referred to as Internet of Things (“IoT”) (e.g., edge IOT device 705) that may be configured to send and receive information via a network to access cloud computing services or interact with a remote web browser application (e.g., to receive configuration information).

FIG. 7 also illustrates that customer network 702 includes local compute resources 706A-C that may include a server, access point, router, or other device configured to provide for local computational resources and/or facilitate communication amongst networks and devices. For example, local compute resources 706A-C may be one or more physical local hardware devices, such as the auto-mode network infrastructure devices outlined above. Local compute resources 706A-C may also facilitate communication between other external applications, data sources (e.g., 707A and 707B), and services, and customer network 702.

Network infrastructure 700 also includes cellular network 703 for use with mobile communication devices. Mobile cellular networks support mobile phones and many other types of mobile devices such as laptops etc. Mobile devices in network infrastructure 700 are illustrated as mobile phone 704D, laptop computer 704E, and tablet computer 704C. A mobile device such as mobile phone 704D may interact with one or more mobile provider networks as the mobile device moves, typically interacting with a plurality of mobile network towers 720, 730, and 740 for connecting to the cellular network 703.

FIG. 7 illustrates that customer network 702 is coupled to a network 708. Network 708 may include one or more computing networks available today, such as other LANs, wide area networks (“WAN”), the Internet, and/or other remote networks, in order to transfer data between client devices 704A-D and cloud service provider network 710. Each of the computing networks within network 708 may contain wired and/or wireless programmable devices that operate in the electrical and/or optical domain.

In FIG. 7, cloud service provider network 710 is illustrated as a remote network (e.g., a cloud network) that is able to communicate with client devices 704A-E via customer network 702 and network 708. The cloud service provider network 710 acts as a platform that provides additional computing resources to the client devices 704A-E and/or customer network 702. In one implementation, cloud service provider network 710 includes one or more data centers 712 with one or more server instances 714. Cloud service provider network 710 may also include one or more frames or clusters (and cluster groups) representing a scalable compute resource that may implement the techniques of this disclosure. Specifically, disclosed techniques to improve distribution of prescription drugs may be implemented using a cloud-based system for different functional components.

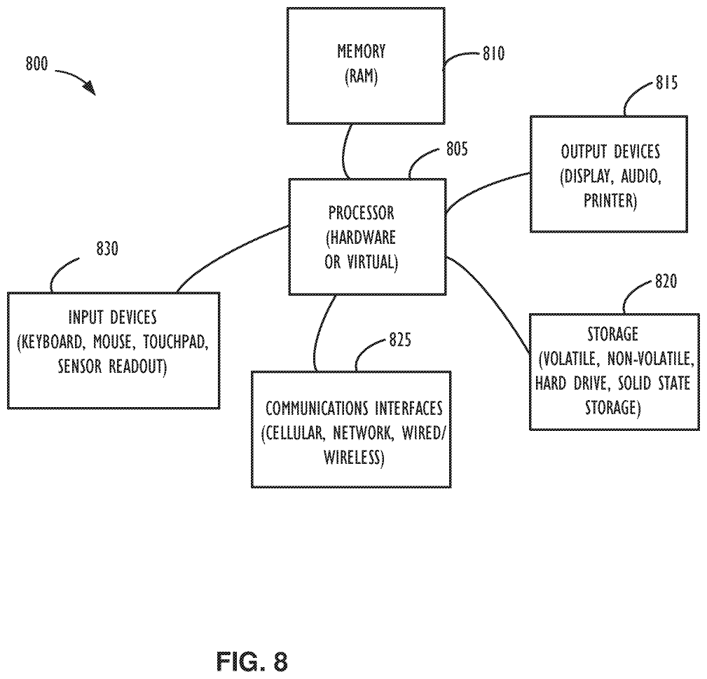

FIG. 8 illustrates a computing device 800 that may be used to implement or be used with the functions, modules, processing platforms, execution platforms, communication devices, and other methods and processes of this disclosure. For example, computing device 800 illustrated in FIG. 8 could represent a client device or a physical server device and include either hardware or virtual processor(s) depending on the level of abstraction of the computing device. In some instances (without abstraction), computing device 800 and its elements, as shown in FIG. 8, each relate to physical hardware. Alternatively, in some instances one, more, or all of the elements could be implemented using emulators or virtual machines as levels of abstraction. In any case, no matter how many levels of abstraction away from the physical hardware, computing device 800 at its lowest level may be implemented on physical hardware.

As also shown in FIG. 8, computing device 800 may include one or more input devices 830, such as a keyboard, mouse, touchpad, or sensor readout (e.g., biometric scanner) and one or more output devices 815, such as displays, speakers for audio, or printers. Some devices may be configured as input/output devices also (e.g., a network interface or touchscreen display).

Computing device 800 may also include communications interfaces 825, such as a network communication unit that could include a wired communication component and/or a wireless communications component, which may be communicatively coupled to processor 805. The network communication unit may utilize any of a variety of proprietary or standardized network protocols, such as Ethernet, TCP/IP, to name a few of many protocols, to effect communications between devices. Network communication units may also comprise one or more transceiver(s) that utilize the Ethernet, power line communication (“PLC”), WiFi®, cellular, and/or other communication methods.

As illustrated in FIG. 8, computing device 800 includes a processing element such as processor 805 that contains one or more hardware processors, where each hardware processor may have a single or multiple processor core. In one implementation, the processor 805 may include at least one shared cache that stores data (e.g., computing instructions) that are utilized by one or more other components of processor 805. For example, the shared cache may be a locally cached data stored in a memory for faster access by components of the processing elements that make up processor 805. In one or more implementations, the shared cache may include one or more mid-level caches, such as level 2 (“L2”), level 3 (“L3”), level 4 (“L4”), or other levels of cache, a last level cache (“LLC”), or combinations thereof. Examples of processors include but are not limited to a central processing unit (“CPU”) a microprocessor. Although not illustrated in FIG. 8, the processing elements that make up processor 805 may also include one or more of other types of hardware processing components, such as graphics processing units (“GPU”), application specific integrated circuits (“ASICs”), field-programmable gate arrays (“FPGAs”), and/or digital signal processors (“DSPs”).

FIG. 8 illustrates that memory 810 may be operatively and communicatively coupled to processor 805. Memory 810 may be a non-transitory medium configured to store various types of data. For example, memory 810 may include one or more storage devices 820 that comprise a non-volatile storage device and/or volatile memory. Volatile memory, such as random-access memory (“RAM”), can be any suitable non-permanent storage device. The non-volatile storage devices 820 can include one or more disk drives, optical drives, solid-state drives (“SSDs”), tap drives, flash memory, read only memory (“ROM”), and/or any other type of memory designed to maintain data for a duration of time after a power loss or shut down operation. In certain instances, the non-volatile storage devices 820 may be used to store overflow data if allocated RAM is not large enough to hold all working data. The non-volatile storage devices 820 may also be used to store programs that are loaded into the RAM when such programs are selected for execution.

Persons of ordinary skill in the art are aware that software programs may be developed, encoded, and compiled in a variety of computing languages for a variety of software platforms and/or operating systems and subsequently loaded and executed by processor 805. In one implementation, the compiling process of the software program may transform program code written in a programming language to another computer language such that the processor 805 is able to execute the programming code. For example, the compiling process of the software program may generate an executable program that provides encoded instructions (e.g., machine code instructions) for processor 805 to accomplish specific, non-generic, particular computing functions. Sets of computer instructions to program a computer may be referred to as functional modules or simply modules that execute on a processor of the computer system.

After the compiling process, the encoded instructions may then be loaded as computer executable instructions or process steps to processor 805 from storage device 820, from memory 810, and/or embedded within processor 805 (e.g., via a cache or on-board ROM). Processor 805 may be configured to execute the stored instructions or process steps in order to perform instructions or process steps to transform the computing device into a non-generic, particular, specially programmed machine or apparatus. Stored data, e.g., data stored by a storage device 820, may be accessed by processor 805 during the execution of computer executable instructions or process steps to instruct one or more components within the computing device 800.

A user interface (e.g., output devices 815 and input devices 830) can include a display, positional input device (such as a mouse, touchpad, touchscreen, or the like), keyboard, or other forms of user input and output devices. The user interface components may be communicatively coupled to processor 805. When the output device is or includes a display, the display can be implemented in various ways, including by a liquid crystal display (“LCD”) or a cathode-ray tube (“CRT”) or light emitting diode (“LED”) display, such as an organic light emitting diode (“OLED”) display. Persons of ordinary skill in the art are aware that the computing device 800 may comprise other components well known in the art, such as sensors, powers sources, and/or analog-to-digital converters, not explicitly shown in FIG. 8.

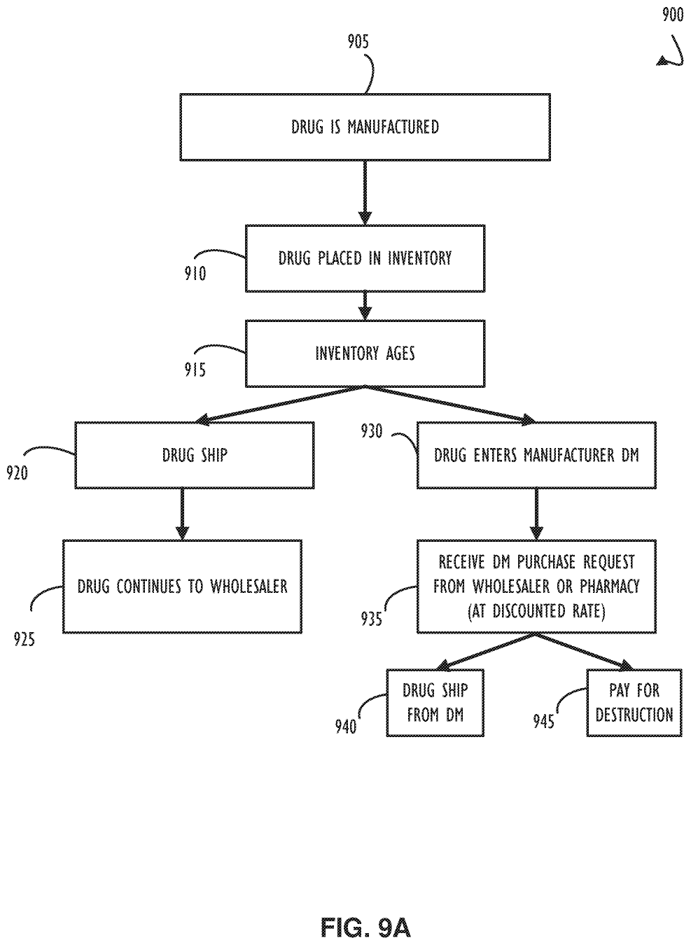

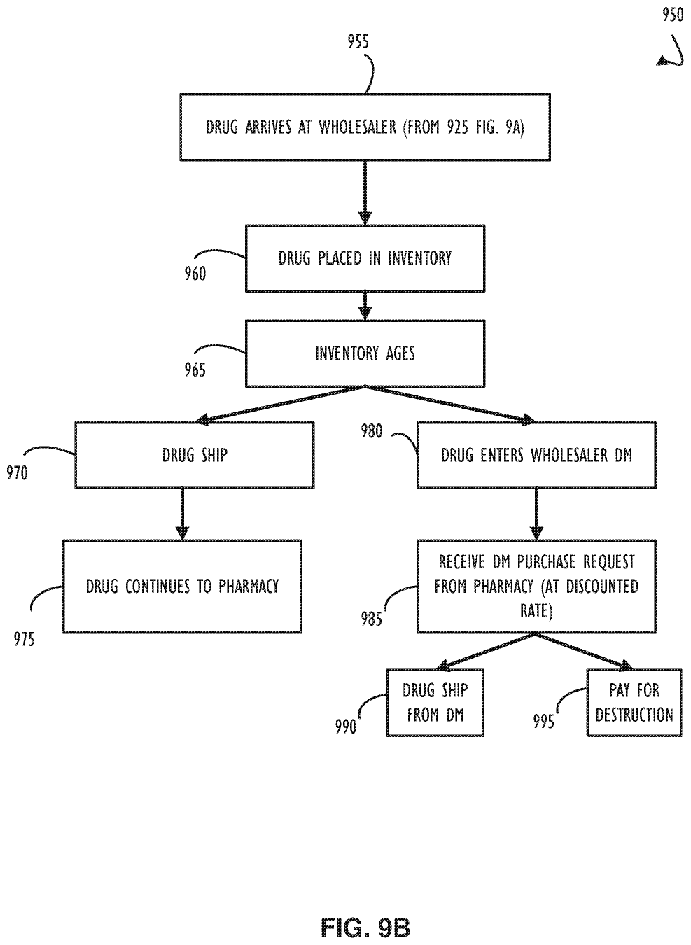

Having discussed the overall capabilities of different computer implementations for embodiments of a prescription drug purchase system, reference is made to FIG. 9A to illustrate a drug lifecycle with respect to a drug Manufacturer and to FIG. 9B to illustrate a drug lifecycle with respect to a drug wholesaler. FIG. 9A illustrates a lifecycle 900 for a drug as may take place at a drug Manufacturer. Beginning at block 905, a drug is manufactured and placed into inventory at the Manufacturer as indicated at block 910. After placement in inventory, time passes and the drug ages as indicated at block 915. In a typical lifecycle a drug will ship from inventory as indicated at block 920 and continue to a wholesaler as indicated at block 925.

In contrast to the above explained typical flow, disclosed embodiments provide systems and methods to handle at least one alternate path through lifecycle 900. Specifically, if the drug does not ship for a period of time (different periods of time may exist for different types of drugs and possibly different manufactured “lots”), the drug may enter a DM at the Manufacturer as indicated at block 930. As explained above, there may be many reasons that a drug enters a DM. In this example, a drug is designated to enter a DM because it has been in inventory and aged to a point where contract terms prevent shipment to a drug wholesaler through the flow that includes block 920. Even though the drug has aged, it may still have useful life and disclosed systems try to make that drug available for a variety of reasons (e.g., cost, environment, efficiency, etc.). Accordingly, upon entry of the drug to a Manufacturer DM (block 930), the Manufacturer may publicize via disclosed systems that this and other similar drugs in the DM are available for purchase through an alternate distribution path. Block 935 indicates that a request (likely responsive to publication in the disclosed distribution system) may arrive to purchase the specific drug from the DM (likely at a discounted rate). If a request for purchase is not made during the useful time period for a drug, block 945 indicates that the Manufacturer may be forced to pay for proper destruction of the drug. Proper destruction of drugs typically includes overhead of tracking and has environmental considerations.

To prevent destruction and reduce the undesirable consequences of destruction mentioned above, disclosed systems increase the likelihood of proper drug usage without resorting to destruction. As illustrated in lifecycle 900, upon receipt of the purchase request within the usable time period of a drug in the DM, flow of lifecycle 900 continues to block 940 where the drug may ship from the DM to the requesting entity. In this example, a requesting entity to the Manufacturer may be either a wholesaler or a Pharmacy. If purchase is made directly between a Manufacturer and a Pharmacy, tracking of lot numbers and specific instances of drugs may be enhanced such that if a recall were to happen, it would likely be easier to track the actual drug and improve public safety. This improved tracking is, in part, because the Manufacturer would have direct knowledge of the actual Pharmacy that received the drug and the Pharmacy would have records to indicate the actual patient that received the drug.

Referring now to FIG. 9B, lifecycle 950 is similar to that of lifecycle 900 but is presented from the perspective of the drug wholesaler as opposed to the drug Manufacturer. Lifecycle 950 begins at block 955 where a drug may arrive at a wholesaler from a drug Manufacturer such as illustrated in block 925 of FIG. 9A. Block 960 indicates that the drug is placed into wholesaler inventory and begins to age as indicated at block 965. While the drug is in inventory, a request for purchase may be received and the drug may ship (block 970) to a Pharmacy as indicated at block 975. This branch of blocks 970 and 975 may be considered to represent a typical flow of a drug without entry of that drug into a DM.

In contrast to the typical flow (e.g., without use of a DM) outlined above for the wholesaler, a drug may age while in inventory to a point where that drug is no longer viable (e.g., based on contract terms) for shipment via the above path. Even though the drug may not be viable with respect to contract terms, the drug may still have useful life and accordingly enters a DM at the wholesaler as indicated at block 980. While in the DM, the wholesaler may take similar actions to publicize availability of a drug at a discounted rate (e.g., via the disclosed prescription drug distribution system) as have been explained throughout this disclosure (e.g., similar to Manufacturer in FIG. 9A). Block 985 indicates that a request for purchase from the DM at the wholesaler may arrive from a Pharmacy (e.g., request via the disclosed system) and the drug may ship from the DM as indicated at block 990. However, if no request for purchase is received, the wholesaler may have to pay for proper destruction of the drug as indicated at block 995. Drug destruction by a wholesaler has similar concerns as those listed above with respect to destruction by a Manufacturer.

It should be further noted that at the Pharmacy level there may be an implementation of a Pharmacy level DM where the DM may be shared across retail stores within the same chain or across Pharmacies within a geographical region. In this manner, the DM may be efficiently used at the Pharmacy level to further reduce cost and waste of prescription drugs. Still further, environmental impacts due to drug destruction may be minimized when more efficient use of drugs (rather than destruction) is realized.

Certain terms have been used throughout this description and claims to refer to particular system components. As one skilled in the art will appreciate, different parties may refer to a component by different names. This document does not intend to distinguish between components that differ in name but not function. In this disclosure and claims, the terms “including” and “comprising” are used in an open-ended fashion, and thus should be interpreted to mean “including, but not limited to. . . . ” Also, the term “couple” or “couples” is intended to mean either an indirect or direct wired or wireless connection. Thus, if a first device couples to a second device, that connection may be through a direct connection or through an indirect connection via other devices and connections. The recitation “based on” is intended to mean “based at least in part on.” Therefore, if X is based on Y, X may be a function of Y and any number of other factors.

The above discussion is meant to be illustrative of the principles and various implementations of the present disclosure. Numerous variations and modifications will become apparent to those skilled in the art once the above disclosure is fully appreciated. It is intended that the following claims be interpreted to embrace all such variations and modifications.

What is claimed is:

1. A computer system configured to perform server side actions as part of a distributed, network based, computer application, the computer system comprising:
one or more processing units, memory communicatively coupled to the one or more processing units, and one or more network interfaces communicatively coupled to the memory and to the one or more processing units, wherein the memory comprises computer instructions to cause the one or more processing units to:
receive product offering information via the one or more network interfaces from one or more drug manufacturers, the product offering information comprising at least a set of information about a drug available in a drug morgue (“DM”) maintained by the one or more drug manufacturers, wherein the product offering information comprises information pertaining to remaining useable time period of the drug and discounted pricing information for purchase of the drug from the DM;
provide at least a subset of the product offering information to a purchasing entity, the purchasing entity being one of the one or more drug wholesalers and pharmacies, via an independent interface to the computer system from the one or more drug wholesalers and pharmacies;
receive a request for purchase from the purchasing entity; and
process the request for purchase to accept the request for purchase and initiate shipment of the drug from the drug manufacturer to the purchasing entity.

2. The computer system of claim 1, wherein the computer system is implemented as a cloud based computer system having independent interfaces for each of: the one or more drug manufacturers, the one or more drug wholesalers; and the pharmacies.

3. The computer system of claim 1, wherein the memory further comprises computer instructions to cause the one or more processing units to:
register each of the one or more drug manufacturers, the one or more drug wholesalers; and the pharmacies as a purchasing entity to prevent providing the product offering information to an unregistered entity, wherein registration includes collecting purchasing entity information.

4. The computer system of claim 3, wherein the purchasing entity information includes at least one of: class of trade information regarding the purchasing entity, purchase volume information regarding the purchasing entity, contact information regarding the purchasing entity, and drug purchase licensing information regarding the purchasing entity.

5. The computer system of claim 4, wherein the memory further comprises computer instructions to cause the one or more processing units to:
adjust the discounted pricing information based on a class of trade with respect to the purchasing entity prior to providing the at least a subset of the product offering information to the purchasing entity, wherein different purchasing entities receive different discounted pricing information.

6. The computer system of claim 1, wherein the memory further comprises computer instructions to cause the one or more processing units to receive a request for purchase from a purchasing entity including instructions to receive multiple requests for purchase from multiple purchasing entities prior to accepting one of the multiple requests for purchase of the drug.

7. The computer system of claim 6, wherein the memory further comprises computer instructions to cause the one or more processing units to facilitate an auction determination across the multiple requests for purchase of the drug to determine the one of the multiple requests for purchase of the drug.

8. The computer system of claim 1, wherein the memory further comprises computer instructions to cause the one or more processing units to maintain tracking information with respect to the shipment of the drug to provide information pertaining to end-user purchase of the drug.

9. The computer system of claim 1, wherein the memory further comprises computer instructions to cause the one or more processing units to maintain information regarding DM purchases across a set of related pharmacies to implement a Pharmacy level DM.

10. The computer system of claim 1, wherein the memory further comprises computer instructions to cause the one or more processing units to:
automatically receive the product offering information from an inventory management system; and
automatically reduce the discounted pricing information based on reduction of the remaining useable time period of the drug.

11. A computer system configured to perform server side actions as part of a distributed, network based, computer application, the computer system comprising:
one or more processing units, memory communicatively coupled to the one or more processing units, and one or more network interfaces communicatively coupled to the memory and to the one or more processing units, wherein the memory comprises computer instructions to cause the one or more processing units to:
receive product offering information via the one or more network interfaces from one or more drug wholesalers, the product offering information comprising at least a set of information about a drug available in a drug morgue (“DM”) maintained by the one or more drug wholesalers, wherein the set of information from the one or more drug wholesalers comprises information pertaining to remaining useable time period of the drug and discounted pricing information for purchase of the drug from the DM;
provide at least a subset of the product offering information to one or more pharmacies via an independent interface to the computer system from the one or more pharmacies;
receive a request for purchase from a purchasing entity, the purchasing entity being one of the one or more pharmacies; and
process the request for purchase to initiate shipment of the drug from the drug wholesaler to the purchasing entity.

12. The computer system of claim 11, wherein the computer system is implemented as a cloud based computer system having independent interfaces for each of: the one or more drug wholesalers; and the pharmacies.

13. The computer system of claim 11, wherein the memory further comprises computer instructions to cause the one or more processing units to:
register each of the one or more drug wholesalers; and the pharmacies as a purchasing entity to prevent providing the product offering information to an unregistered entity, wherein registration includes collecting purchasing entity information.

14. The computer system of claim 13, wherein the purchasing entity information includes at least one of: class of trade information regarding the purchasing entity, purchase volume information regarding the purchasing entity, contact information regarding the purchasing entity, and drug purchase licensing information regarding the purchasing entity.

15. The computer system of claim 14, wherein the memory further comprises computer instructions to cause the one or more processing units to:
adjust the discounted pricing information based on a class of trade with respect to the purchasing entity prior to providing the at least a subset of the product offering information to the purchasing entity, wherein different purchasing entities receive different discounted pricing information.

16. The computer system of claim 11, wherein the memory further comprises computer instructions to cause the one or more processing units to receive a request for purchase from a purchasing entity including instructions to receive multiple requests for purchase from multiple purchasing entities prior to accepting one of the multiple requests for purchase of the drug.

17. The computer system of claim 16, wherein the memory further comprises computer instructions to cause the one or more processing units to facilitate an auction determination across the multiple requests for purchase of the drug to determine the one of the multiple requests for purchase of the drug.

18. The computer system of claim 11, wherein the memory further comprises computer instructions to cause the one or more processing units to maintain tracking information with respect to the shipment of the drug to provide information pertaining to end-user purchase of the drug.

19. The computer system of claim 11, wherein the memory further comprises computer instructions to cause the one or more processing units to maintain information regarding DM purchases across a set of related pharmacies to implement a Pharmacy level DM.

20. The computer system of claim 11, wherein the memory further comprises computer instructions to cause the one or more processing units to:
automatically receive the product offering information from an inventory management system; and
automatically reduce the discounted pricing information based on reduction of the remaining useable time period of the drug.
